(19) United States
(12) Patent Application Publication
(10) Pub. No.: US 2024/0255395 A1
(43) Pub. Date: Aug. 1, 2024

(54) METHODS, DEVICES AND SYSTEMS FOR SEPARATING BIOLOGICAL ANALYTES FROM SAMPLES

(71) Applicant: REGENTS OF THE UNIVERSITY OF MINNESOTA, Minneapolis, MN (US)

(72) Inventors: Abdennour Abbas, Lauderdale, MN (US); Akli Zarouri, St. Paul, MN (US)

(21) Appl. No.: 18/565,948

(22) PCT Filed: Jun. 3, 2022
(86) PCT No.: PCT/US2022/032086

§ 371 Date: Nov. 30, 2023

Related U.S. Application Data
(60) Provisional application No. 63/196,542, filed on Jun. 3, 2021.

(51) Int. Cl. G01N 1/34; C12N 15/10

(52) U.S. Cl. CPC G01N 1/34; C12N 15/1017

(57) ABSTRACT

A device for separating biological entities from a fluid sample, the device comprising: porous material comprising bound material, wherein the bound material functions to separate the biological entity from the fluid matrix. Devices including such porous media, methods of using such porous media and methods of making such porous media are also included herein.

### CROSS-REFERENCE TO RELATED APPLICATION

This application claims the benefit of U.S. Provisional Patent Application No. 63/196,542, filed Jun. 3, 2021, which is incorporated herein by reference in its entirety.

### GOVERNMENT FUNDING

This invention was made with government support under CBET-2028909 awarded by the National Science Foundation (NSF), and 2021-33530-34609 awarded by the National Institute of Food and Agriculture, USDA. The government has certain rights in the invention.

### SUMMARY

This disclosure describes devices for separating biological entities from a fluid sample, the device comprising: porous material comprising bound material, wherein the bound material functions to separate the biological entity from the fluid sample.

Additionally disclosed are devices for separating biological entities from a fluid sample, the device comprising: porous material comprising bound nanoparticles bonded to a bound material, wherein the bound material functions to separate the biological entity from the fluid sample.

Also disclosed are methods of forming filtration media, the method comprising: contacting a sheet of porous material having a matrix and pores within the matrix, with a functionalization solution, the functionalization solution comprising a material selected from: an organic material, a biological entity, an inorganic material, or a combination thereof and a solvent; and forcing the functionalization solution through the pores of the porous material in a direction that is substantially parallel to the direction that a fluid sample is to be filtered using the filtration media.

Also disclosed are devices for separating biological entities from a fluid sample, the device comprising: at least one filter, the at least one filter comprising a sheet of porous material comprising bound material; an inlet; an outlet, wherein the fluid sample comprising the biological entities and a fluid matrix are introduced via the inlet of the filter, the fluid sample contacts the porous material of the filter wherein the bound material functions to separate the biological entities from the fluid matrix and the fluid matrix exits the device via the outlet.

Also disclosed are methods of separating biological entities from a fluid sample, the method comprising: contacting the fluid sample with a porous material, the porous material comprising a matrix having surface area and pores, the surface area of the porous material comprising bound material; removing at least some of the fluid matrix from the porous material; and retaining at least some of the biological entities within or in contact with the bound material.

Also disclosed are methods and devices wherein the fluid sample can be forced through the porous material using a vacuum pump. Also disclosed are methods or devices that further comprise a syringe, a collection tube, or a fluidic system connected to the inlet of the device. Also disclosed are methods or devices that further comprise a syringe, a collection tube, or a fluidic system connected to the outlet of the device. Also disclosed are methods or devices that further comprise at least one chamber surrounding the filter on the outlet side, the at least one chamber being used to contain solutions for sample lysis, bonding, preservation, extraction, filtration, or any combination thereof.

Also disclosed are methods or devices wherein the porous media is formed by drawing, forcing, or some other combination thereof a functionalization solution through a sheet of the porous media in a direction substantially parallel to the direction in which a fluid sample will be drawn through the sheet of the porous media.

Disclosed herein are a porous media and devices that include such porous media (e.g., filters) that have been functionalized with metal oxide nanoparticles that bind an organo-silicon compound. Such porous media and devices can be used, for example, for applications in biological separations.

Disclosed herein are porous media and devices that include such porous media (e.g., filters) that have been functionalized directly with an organic or inorganic compound. In some embodiments, the porous media was functionalized using a method that is referred to herein as crossflow functionalization. Such porous media and devices can be used, for example, for applications in biological separations.

Disclosed herein are methods of forming porous media including forcing a functionalization solution across the porous media using a pumping system, for example. Such methods can be referred to herein as “crossflow functionalization”. Crossflow functionalization can be utilized for the functionalization of organic or inorganic porous media with materials in order to provide the porous media with new functionalities. Such methods can be utilized to form porous media that can be utilized, for example, in applications such as chemical or biological separations, biological catalysis processes, energy harvesting applications, storage and conversion applications, gas and fluid filtration applications, antimicrobial protection applications, and sensing and biosensing applications.

Disclosed herein are biocollection and bio-extraction systems that combine both sample collection and separation of biological targets (e.g., nucleic acids, cells, tissues, microorganisms, viruses, prions, biomolecules, and molecules) from small or large volumes of fluid samples.

Also disclosed herein are devices that include disclosed porous media and are similar to currently utilized spin columns, and/or other filtration systems containing disclosed porous media in the form of filters.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 shows the silane structures used to functionalize porous media (3) through use of a nanoparticle (2), for example. (A) X is a hydrolysable (eg. Oxysilane) or metal interacting group (amine, thiol), and the attachment of the functionalization agent to the porous media relies on covalent binding formed by a hydrolysis reaction. (B) Silanization using a hydrolysable (e.g., oxysilane) or metal interacting group (amine, thiol). The attachment of the functionalization agent to the porous media relies on covalent binding formed by a hydrolysis reaction. (C) Functionalization of porous media (3) with a cationic compound or polymer. R is the radical group of an amine terminated compound or polymer (e.g., polylysine, or polyallylamine). The attachment of the functional groups to the porous media relies on electrostatic interactions.

FIG. 2 shows a comparison of conventional bioseparation filters (left) and disclosed porous media that includes nanoparticles (middle) and functional polymers or only functional polymers (right). Any of the disclosed porous media can capture biological targets through high chemical affinity with the functional polymers, while conventional filters mainly rely on pore size (size exclusion) and physical separation.

FIG. 3 shows scanning electron microscopy (SEM) images of a cellulose filter paper before and after silanization. (A) Before silanization. (B) After silanization.

FIG. 4 shows a system for the silanization of filters. The elements noted in FIG. 4 include a tank 10 containing the silanization solution 12, a filter to be functionalized 14, flow direction of the silanization solution 16, and vacuum pump 18 aspiring the silanization solution through the filter allowing its efficient functionalization.

FIG. 5 shows a spin column based on silanized cellulose filters. A spin column 20 can contain one or multiple silanized filters 14 and each filter 14 can have a different pore size, a different material, a different functionalization, or any combination thereof.

FIG. 6 shows exemplary filter holders. (A) Filter 14 is positioned between two filter supports 22 that include a lumen 24 through which fluid can flow 26. (B) An alternative exemplary configuration in which the filter 14 is a single filter support 22 having a lumen 24 through which fluid can flow 26. (C) Another alternative exemplary configuration in which the filter 14 is positioned between two filter supports 22 that include a lumen 24.

FIG. 7. Exemplary biocollection and bioseparation devices. (A) Side view of device 30, which includes a sample collection tube 32, a filter holder 34, and two collection tubes, and a flow-through collection tube 36. (B) Exploded view of device 30, which includes a sample collection tube 32, a filter holder 34, and two collection tubes, and a flow-through collection tube 36.

FIG. 8. shows combined pieces of a disclosed Biosampler 38. (A) A photograph of an exemplary embodiment of the Biosampler 38. (B) A cross-section view of the Biosampler 38 showing a first reservoir 40, a second reservoir 42, and a centrifugation tube 44. (C) A cross-section view of the Biosampler 38, showing a body 50 that includes a cap 58, filters 54, a filter support 56, an O-ring 52, and a plug. A syringe 62 is shown.

FIG. 9A-FIG. 9W show specific filters, devices, and components thereof that can include or can be used with the disclosed porous media.

FIG. 10 shows loading capacity of the silanized filter (Nanosorbent) as compared to conventional filters.

FIG. 11 shows Koi Herpesvirus environmental DNA (eDNA) capture and recovery rate from aquaculture water using a silanized filter (Nanosorbent) versus a cellulose filter or a nitrate cellulose filter.

FIG. 12 shows a comparison of cycle threshold (CT) values obtained with the commercial kits versus those obtained with kits using the silanized filter technology described herein (Frontline Biotech. Ct).

FIG. 13 shows comparisons of the detection limits and CT values of the RNA extraction kits from commercial kit (Qiagen) and the kit using the silanized filter technology described herein (Frontline Biotech. Ct). The x axis is in logarithmic scale. This figure is also used to calculate the analytical sensitivity from the slope of the linear fit.

FIG. 14 shows a design of the nucleic acid collection and isolation system for water samples. The system includes a body 50 that contains silanized filters 54 and a spin column 20 that also includes a silanized filter 54.

FIG. 15 shows a workflow of nucleic acid collection and isolation from the collection and filtration of large volumes of water samples (up to 3 L) in the field using an exemplary embodiment of Biosampler 38 to the extraction of pure and concentrated nucleic acids into 10-100 μL sample using the spin column. Both the cartridge and the spin column use the nucleic acid sorbent. This workflow results in a sample that can be used for RT-PCR analysis, sequencing, or barcoding.

FIG. 16 shows a portable device 70 for airborne DNA collection, concentration, extraction, and isolation. The platform includes a housing unit 72 that includes an air pump/fan providing directional air flow 74, collection cartridge 76 containing eDNA nanosorbents 78, an adapter 80 connecting the cartridge to the air flow, and a moisture dispenser 82 to enhance DNA binding to the nucleic acid sorbents.

FIG. 17 shows a workflow of airborne nucleic acid collection and isolation using an air circulation system and a cartridge and a spin column containing nucleic acid sorbents, from the collection and filtration of large volumes of ambient air in the field to the extraction of pure and concentrated nucleic acids into 10-100 μL sample in the laboratory. This workflow results in a sample that can be used for qPCR analysis, profiling, sequencing, or barcoding (Option A). Alternatively (Option B), the recovered cartridge from the air filtration can be used directly to extract the DNA for Rapid DNA analysis or automatized separation and isolation using commercial magnetic beads-based systems.

### DETAILED DESCRIPTION OF ILLUSTRATIVE EMBODIMENTS

This disclosure relates to the development of a novel type of filter that can be used for a variety of biological separations or bioseparation including nucleic acid extraction, protein separation, microbial separation of viruses and other microorganisms, and/or cell and tissue separation. Disclosed herein are the following innovations:

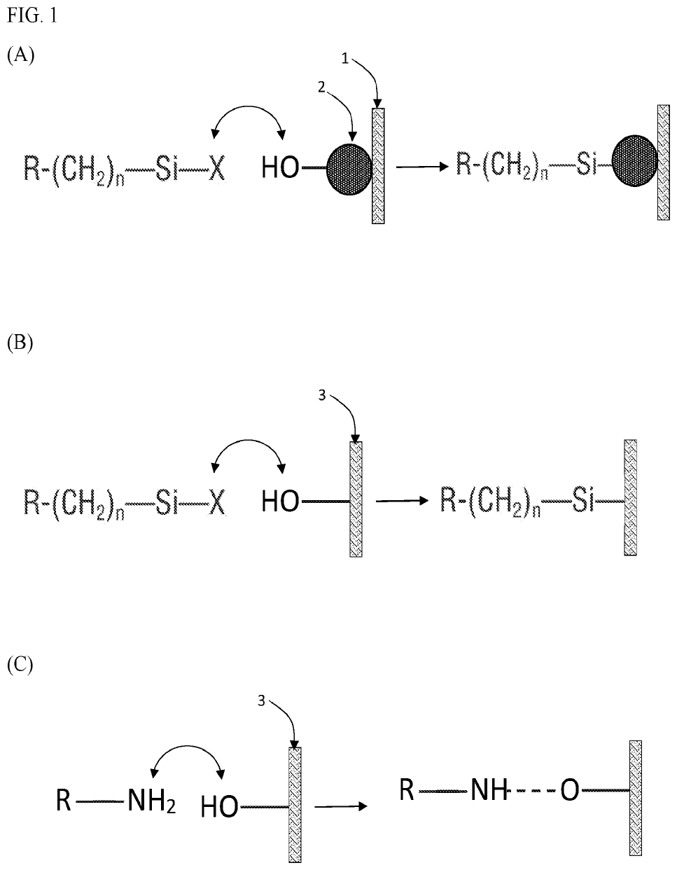

Porous filters functionalized with metal-oxide nanoparticles, organo-silicon compounds, polymeric compounds, organic compounds, or combinations thereof for biological separations (FIG. 1A)

Porous filters functionalized directly with organic polymers such as organo-silicon compounds, functional polymers, biomolecules, or chemical compounds (FIG. 1B and FIG. 1C), to provide high affinity to biological entities and high filtration flow rates for biological separations.

Processes and methods for functionalizing porous media and filters such as cellulose paper with organic or inorganic compounds. The process is referred to herein as “crossflow functionalization.” The process includes drawing or forcing a functionalization solution through the filter media, in a direction parallel to the ultimate direction of flow of a fluid sample to be processed using the filter media. As used herein, the term “fluid” refers to a substance that readily flows under ambient conditions. A fluid may be a liquid or a gas, or a combination thereof.

Biocollection and bioseparation systems and filter holders to enable the processing of large-volume biological samples, such systems can be referred to herein as “Biosampler” or “eBiosampler.”

Current commonly used technologies for bioseparations rely on magnetic bead-based separation or silica gel spin column-based separation. Both are designed for small volumes (e.g., less than 1 mL). As a result, treatment of large volumes is either impossible or time-consuming and costly. Furthermore, the two techniques present other limitations. For example, magnetic separations typically have limitations on sample volume because magnetic separation requires the use of large and costly amounts of magnetic nanoparticles and reagents to analyze volumes of a few milliliters (with a maximum around 60 mL). Spin columns also can have limitations on sample volume due to the density of the silica gel and requiring centrifugation to pass the sample solution through the column to perform separation.

Today, there are several protocols for environmental DNA separation. One of the key steps in these protocols to retrieve DNA from the surface of the filter is to open the filter holder, remove the filter and cut it into small pieces. Next, small pieces of the filter are placed in a tube before starting the DNA extraction using a commercial extraction kit. This process is time-consuming and is also subject to sample contamination which can result in false positives or degradation of the target biological material. Additionally, the capture efficiency and limits of detection of these processes are low. Both the magnetic and silica gel separation have limited capture efficiencies, particularly for nucleic acids: typically, less than 40% of nucleic acid are captured and purified. As a result, the limit of detection for subsequent analytical techniques such as PCR is high, resulting in false negative and false positive results. Thus, current commercial solutions recover and separate free nucleic acid in water, air, or other fluids inefficiently. Further, many filters are only used to separate and recover cells and tissues, then cause cell lysis to recover cellular nucleic acids. Free/extracellular nucleic acids represent an important information that is lost in this process.

In contrast, this disclosure describes filters and systems that efficiently capture free environmental DNA (eDNA) and efficiently capture total DNA contained in cells and tissues. The nucleic acid capture efficiency is made possible by the buffers, sorbents, and/or holding filters described herein.

Advances in genomics, next-generation sequencing, machine learning, and bioinformatics have enabled new applications for DNA analysis. Some of these applications include inferring phenotypic traits or physical appearance from crime scene DNA, mitochondrial DNA sequencing for assessing ancestral origin and identifying human remains, microbiome analysis for profiling, and the use of DNA methylation. As DNA identification techniques advance, DNA collection techniques must similarly improve and become more sensitive. Traditionally, forensic DNA has been primarily collected from visible or concentrated DNA evidence from blood, saliva, semen, skin, or bone fragments.

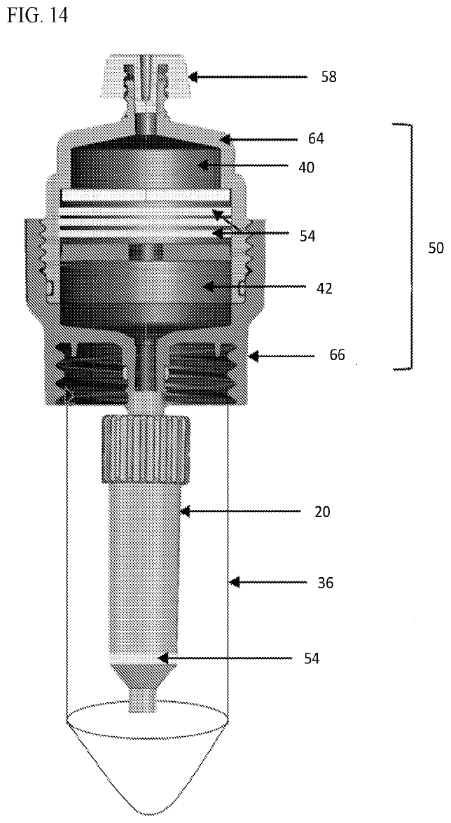

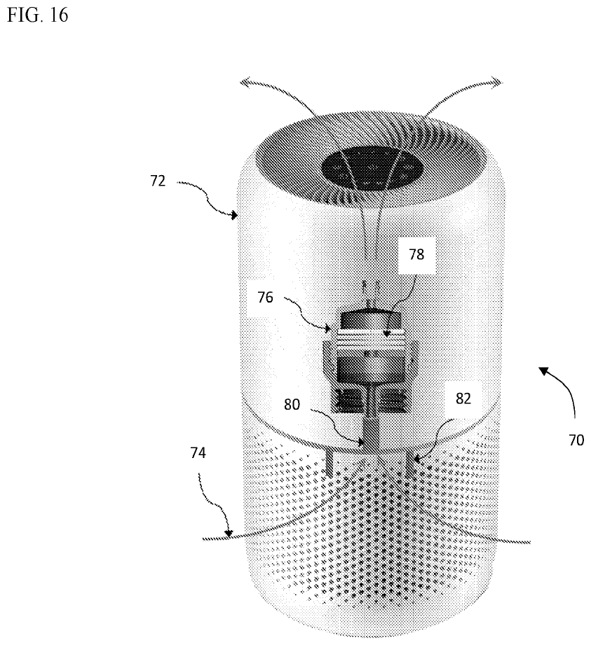

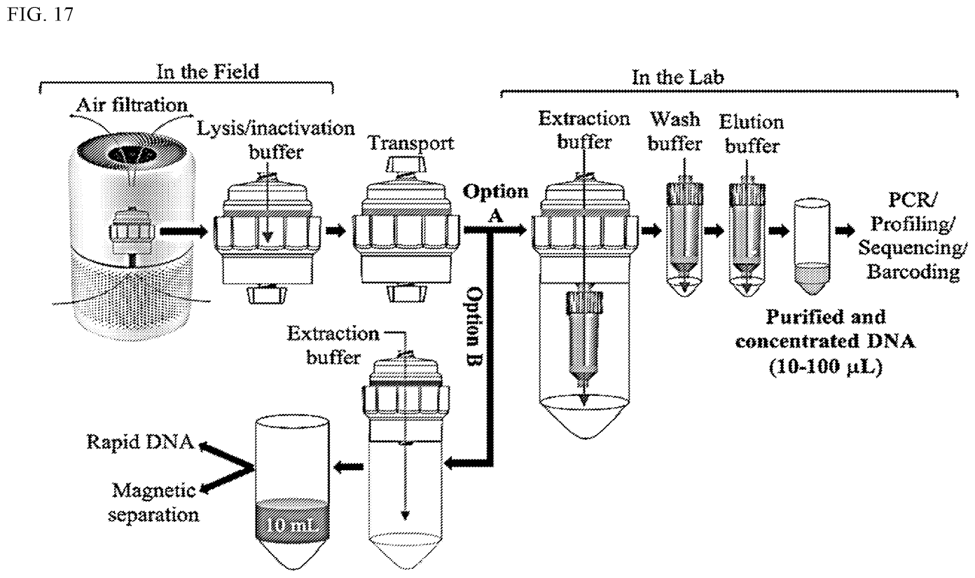

One of the most overlooked sources of (diluted) information and evidence is air. Airborne DNA is particularly important when there is a lack of visible/concentrated evidence such as blood, saliva, semen, or skin. Limited sample collection methods have prevented forensic scientists from commonly using airborne DNA as evidence. This disclosure describes new filter designs suitable for capture of dilute biological material, including DNA, from large fluid volumes. FIG. 16 shows a design of an air filtration system capable of airborne DNA collection and isolation using a cartridge similar to the system described in FIG. 14. A workflow using this system is depicted in FIG. 17.

In some embodiments, the sample processed by the devices or methods of this disclosure is a gas. In some embodiments, the sample may be air. The sample may be a dispersion of particles or dust in air. The sample may be collected using an air filtration unit. The sample may be collected using an air pump or fan. In some embodiments, the sample may be collected using a vacuum. In some embodiments, the devices of this disclosure may include an air filtration unit. In some embodiments, the devices of this disclosure may be able to be coupled to an external air filtration unit. The air filtration unit may be modified to improve sample collection, for example, by incorporation of moisture dispensers.

In some embodiments, the sample is collected in a first location and transported for filtration and analysis at a second location. In some embodiments, the sample may be collected at the first location in a collection unit. In some embodiments, the sample may be collected at the first location in the filtration unit. The filtration unit and/or the collection unit may include material to seal the unit to facilitate transport without loss of the sample. In some embodiments, the device may be sealed by screwcaps.

The present disclosure describes new processes to functionalize filters such as cellulose filters with compounds containing active functional groups, such as organo-silicon containing compounds such as silanes for example; biological entities, organic compounds, or combinations thereof. The resulting filters have high affinity to biological materials (cell, DNA, RNA, microorganisms, viruses), and faster flow rates for filtration, and thus enable rapid and efficient extraction of target biological materials in large volume samples. A new process for filter functionalization and a new filter holder for large volumes are also disclosed.

Cellulose or nitrocellulose paper, glass fiber or metal oxide filters, for example with different pore sizes can be functionalized with a functionalization solution. Illustrative functionalization solutions can include for example, organic or inorganic agents (such as siloxane (e.g., polysiloxane, or oxysilane), or polypetides) following a disclosed process to enable efficient bioseparations with a larger range of sample volumes. Useful agents will have a high affinity for biological targets and are used as receptor or sorbents to capture these targets in reversible fashion.

The filters and systems described herein enable efficient bioseparation using specific chemical affinity rather than relying on pore size (size exclusion) or nonspecific physical separation. The filters and systems described herein enable rapid filtration of large sample volumes (100 uL to 3000 mL), thus allowing one to separate and/or concentrate nucleic acids, viruses, and/or microorganisms from environmental samples such as wastewater. In some cases, the target analytes may be present at extremely low concentrations in an environmental sample so that large sample volumes are necessary to capture a sufficient amount of the target analyte.

The filters and systems described herein also can reduce the number of steps needed to extract biological entities from liquid samples, particularly large-volume samples. As a result, the filters and systems described herein can reduce processing time, cost, and sample contamination.

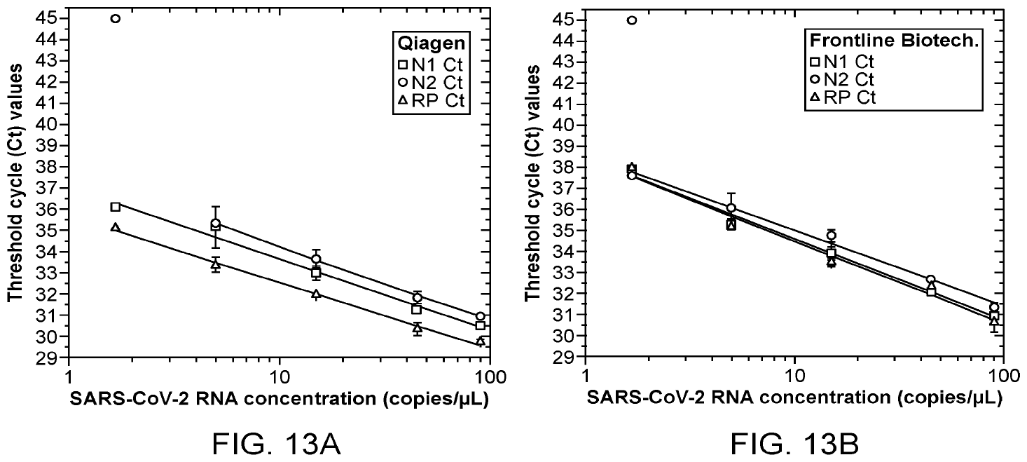

The filters and systems described herein enable one to capture 70% or more of the nucleic acid in a sample during separation, thereby exhibiting higher loading capacity and capture efficiency for nucleic acid than current conventional platforms. The filters and systems described herein therefore offer lower detection limits and better sensitivity than current conventional platforms. For example, the filter described herein enabled the detection of positive samples from COVID-19 patients that were missed by other commercial kits, with high sensitivity (98%) and specificity (100%) (FIG. 13).

The filters and systems described herein also enable one to separate and/or concentrate free nucleic acids in solution, which is currently not possible with available technologies.

Finally, this disclosure describes methods that enable to functionalize filters and other porous media easily and efficiently.

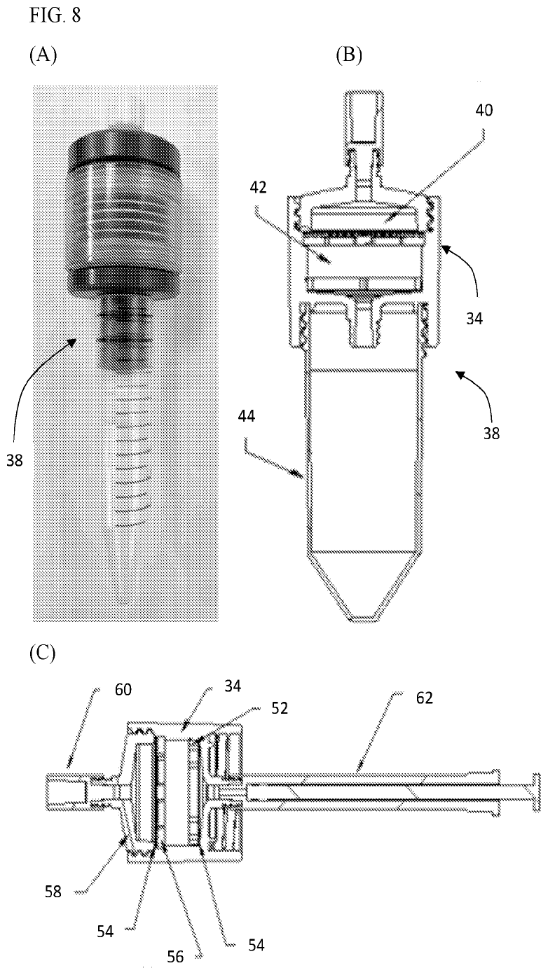

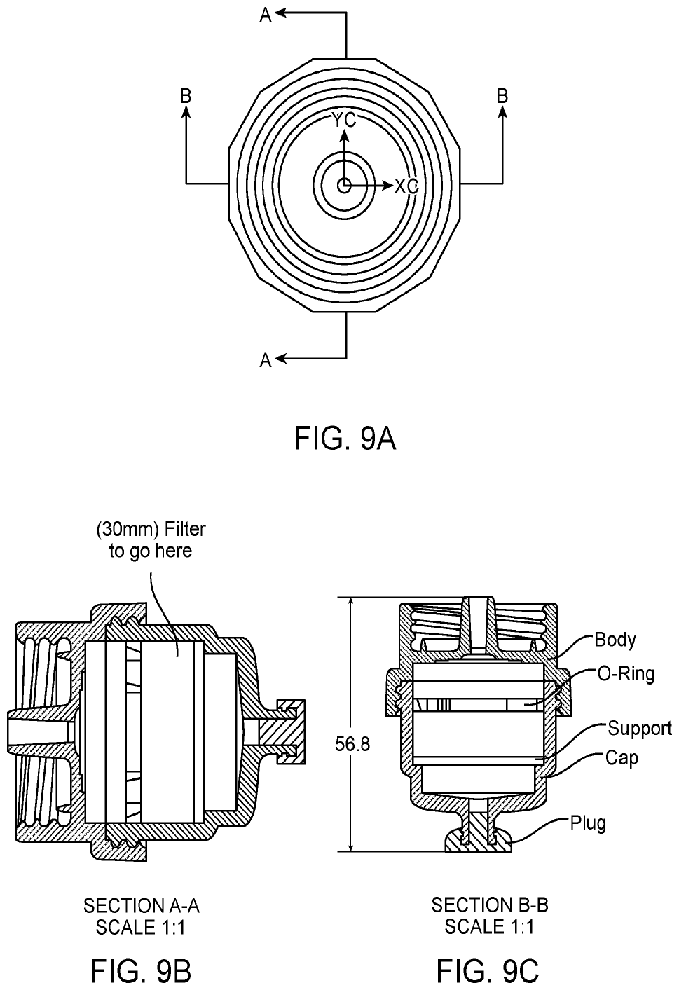

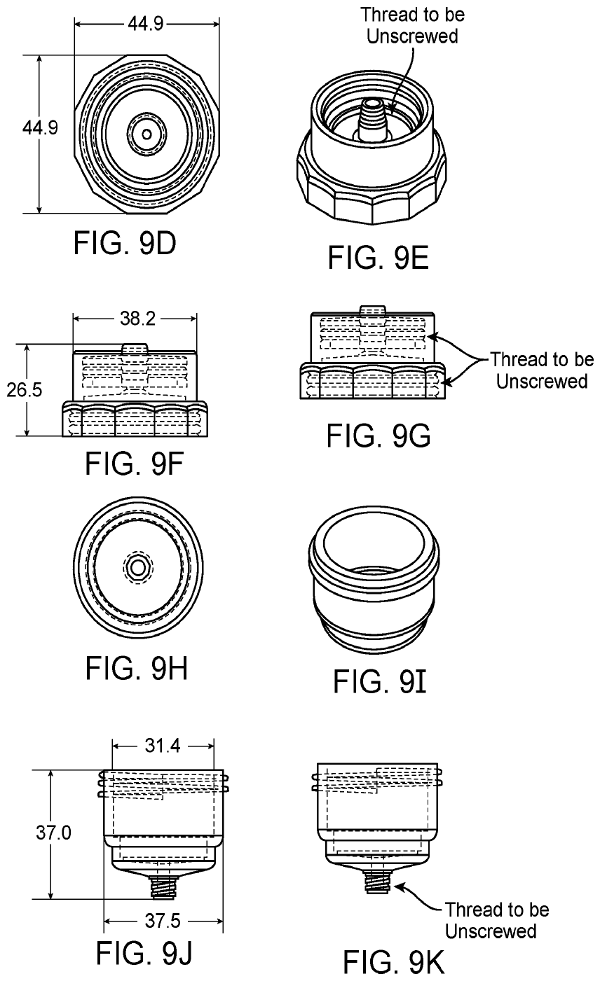

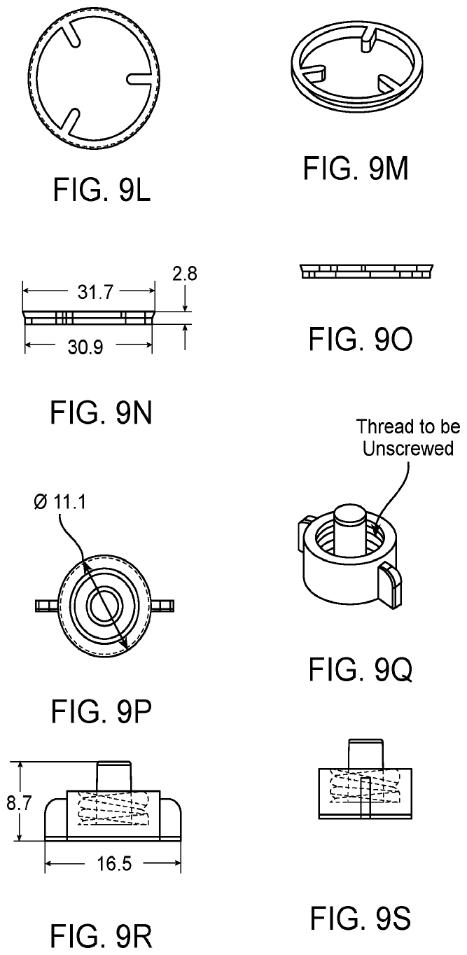

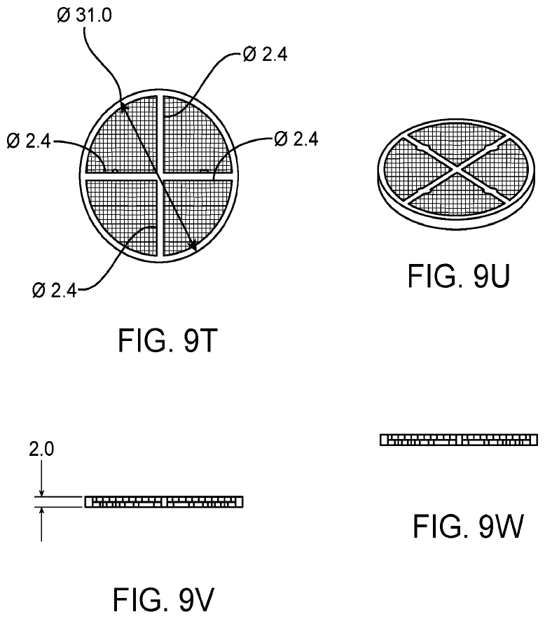

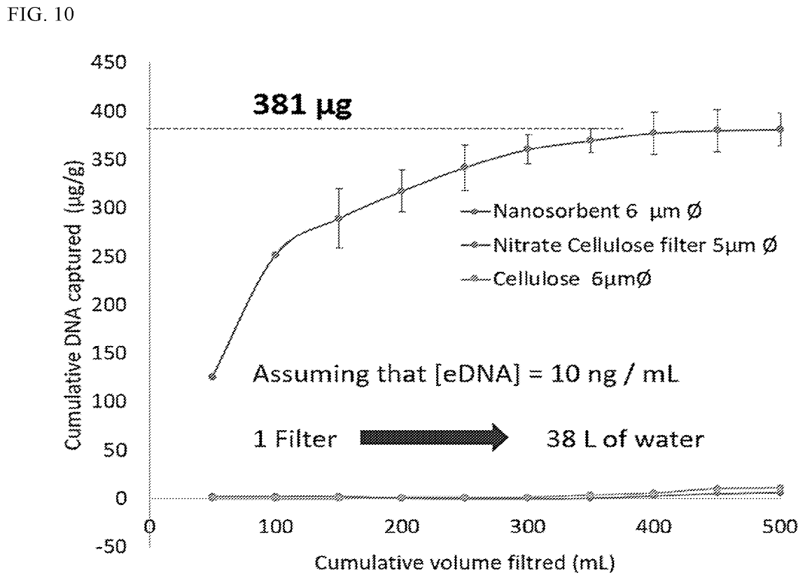

In another aspect, this disclosure describes a device for isolating biological material from a sample. Exemplary embodiments of such a device are shown in FIG. 8 and FIG. 14. Corresponding components of the devices shown in FIG. 8 and FIG. 14 have like reference numerals, but the specific materials of design the components and devices are not limited to the materials and designs illustrated in FIG. 8 and FIG. 14.

The device generally includes a sample collection cartridge 50 and a spin column 20. The sample collection cartridge 50 may include one or more sample chambers 40, 42, one or more layers of sorbent or filter 54, an adapter 66 for attaching the spin column 20 to the collection chamber 50, and a cap 58. In some embodiments, the cap 58 can be designed to provide a fluid-tight seal to reduce (in some cases, even eliminate) the likelihood and/or extent that the sample is contaminated to leaks during transportation. The spin column 20 may include one or more sorbent/filter layers 54 and a collection tube 36.

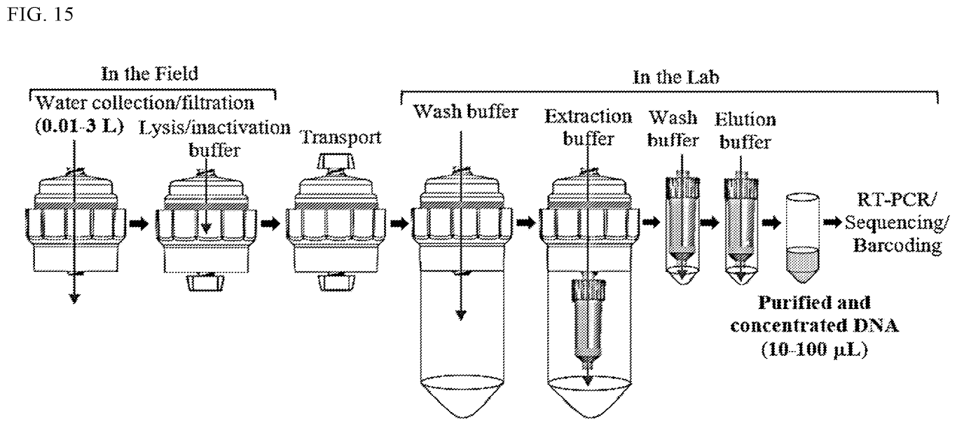

Devices including nucleic acid sorbents may be housed in user-friendly delivery systems. Besides hosting the nanosorbents, the delivery system may address other workflow problems such as sample contamination, hazardous sample transport, cumbersome processing steps, processing time, and cost. The delivery system may consist of a single cartridge that can combine sample collection, filtration, lysis, and inactivation in the field, followed by eDNA isolation and extraction in the lab using a spin column, without the need to have access to the sorbents inside the cartridge. The designed cartridge and spin column may both contain sorbents for the capture of nucleic acids (FIG. 14). A detailed workflow is shown in FIG. 15. The combination of cartridge and spin column enables a faster workflow for nucleic acid collection and isolation.

In another aspect, this disclosure describes a kit including a filtration device. The kit may additionally include buffers for sample processing, material for sample collection, material for sample transport, and instructions. The buffers may include sample collection buffers, wash buffers, elution buffers, extraction buffers, lysis buffers, or a combination thereof. The materials for sample transport may include caps to seal the collection device. The materials for sample transport may include caps to seal the filtration device. The materials for sample collection may include containers (e.g., one or more bags and/or one or more tubes) to hold the filtration and/or sample collection devices. The instruction to use the kit may include steps for column activation, sample collection, sample transport, sample processing, column washing, elution, eluate analysis, or a combination thereof.

In some embodiments, the kit includes the device, instructions, a collection apparatus, material to seal the device, one or more lysis buffers, one or more extraction buffers, one or more wash buffers, and one or more elution buffers. In some embodiments, the collection apparatus is an air filtration unit, a water filtration unit, or a collection tube.

Disclosed methods of functionalization use porous media (e.g., cellulose filters) that are functionalized to have a bound material that functions to separate one or biological entities from a fluid sample. The bound material confers physical, chemical, and mechanical properties to enable more efficient biological separation. Properties that may be optimized include, but are not limited to, affinity and specificity to biological targets (e.g., cells, tissues, viruses, prions, biomolecules, or nucleic acids), filter surface charge to enable faster flow through the filter, filter biocompatibility to enable storage and preservation of biological entities in the solid phase, loading capacity, and filter strength. Filter strength enables one to analyze large volumes with higher flow rates. Additional improved filter properties can include, but are limited to, improved structural stability and chemical resistance of the filters in solutions, optimized pore size of the cellulose paper, optimized filter hydrophobicity or hydrophilicity, and improved affinity for a specific biological target. Affinity for a specific biological target may be optimized by modifying electrostatic charges and interactions of the cellulose paper with the biological targets.

Bioseparation or biological separation, which may be used herein refers to the extraction, isolation, concentration, or any combination thereof, of biological entities or targets. Biological entities are also referred to herein as “biological targets” Exemplary biological entities that can be separated from fluid samples can include, for example, biomolecules (DNA, RNA, proteins, lipids, saccharides or biopolymers or combinations thereof), viruses, prions, and microorganisms (bacteria, fungi, mold), cells, and/or tissues. Biomolecules may include proteins, peptides, amino acids, saccharides, and lipids or a combination thereof. Biopolymers may include polypeptide, oligopeptide, polysaccharides, and oligosaccharides. It should be noted as well that such biological entities can also be functionalized on the porous media to enable one to separate a different biological entity from a fluid sample.

Organic functionalities or functional groups are parts of a molecule that confer a particular reactivity. Exemplary functional groups can include, but are not limited to, acyl, amino, alkane, alkene, alkyne, benzene ring (phenyl), amine, amide, carbonyl, carboxyl, carboxylate, hydroxyl, imine, imino, methyl, nitro, nitrile, anhydride, alcohol, ether, alkyl halide, thiol, aldehyde, ketone, ester, carboxylic acid, sulfide (thioester), epoxide, disulfide, imine, acid chloride, acid anhydride, acyl halide, arene, azo compounds, isocyanate, haloalkane, vinyl, epoxy, methacryloxy, mercapto, phosphate, phosphoryl, phosphoanyhdride, or sulfhydryl. A single molecule can possess one or more functionalities.

Organosilane compounds or organosilane containing compounds are silicon-based molecules that have at least one direct bond between a silicon atom and a carbon atom in the molecule. Oxysilane are organosilane molecules containing at least one direct bond between silicon and oxygen. The terms “organo silane,” “organosilane,” “oxysilane” are used interchangeably here to refer to a carbon and silicon containing monomer.

Oxysilanes can include methoxy, ethoxy, dialkoxy, and trialkoxy types. Oxysilanes can include but are not limited to Tetraethyl orthosilicate, Triethoxy(octyl)silane, Triethoxyoctylsilane, Dodecyltriethoxysilane, n-Octadecyltriethoxysilane, Hexadecyltrimethoxysilane, Triethoxy(ethyl)silane, Isobutyltriethoxysilane, Triethoxymethylsilane, Trimethoxy(octadecyl)silane, (3-Aminopropyl)triethoxysilane, (3-Aminopropyl)trimethoxysilane, (3-Glycidyloxypropyl) trimethoxysilane, Tetraethyl orthosilicate, 3-(Trimethoxysilyl)propyl methacrylate, (3-Aminopropyl)trimethoxysilane, (3-Mercaptopropyl)trimethoxysilane, (3-Glycidyloxypropyl)trimethoxysilane, Trimethoxy(propyl)silane, Trimethoxy(3,3,3-trifluoropropyl)silane, 3-[2-(2-Aminoethylamino)ethylamino]propyl-trimethoxysilane, [3-(2-Aminoethylamino)propyl]trimethoxysilane, Trichlorosilane, 1H, 1H,2H,2H-Perfluorooctyltriethoxysilane, 1H, 1H,2H,2H-Perfluorodecyltriethoxysilane, (3-Glycidyloxypropyl)trimethoxysilane, (3-Glycidyloxypropyl)triethoxysilane, and combinations thereof.

Useful porous materials can include, for example materials having an oxide surface. Such materials can include, for example cellulose, nitrocellulose, glass fiber, metal oxide or combinations thereof. These materials can all be silanized, for example, because they contain hydroxyl groups which attack and displace the alkoxy groups on the silane thus forming a covalent —Si—O—Si— bond. A single filter holder can contain filters made of the same or different materials. Other useful materials can be functionalized to porous materials using other types of bonds besides Si—O bonds.

Useful porous materials can include those having a porosity from 0.1-50 micrometers (μm), preferably 1-12 μm. The porosity of the filters needed depends on the size and concentration of the biological target. Useful devices can include one filter or a plurality of filters. In some embodiments, a useful device can include any of a number of filters. For example, the number of filters needed in each filter holder can range 1-100 filters, preferably 1-20 filters. The number of filters needed depends on the size and concentration of the biological target and pore size of the filters.

The purpose of functionalization of the porous media is to introduce materials or compounds into or onto the porous media to facilitate separation of the biological targets from the fluid sample. In some embodiments, useful material to be functionalized to porous media can include, for example functional groups contained in organic compounds. Useful functional groups can include amine groups, which can impart the ability to draw and maintain certain biological targets into the porous media to enhance the affinity of the filter to biological targets. This functionalization can be performed by the addition of polymers such as oxysilanes (silanization), biopolymers, molecules, or biomolecules. Functionalization can be performed using three different methods, for example.

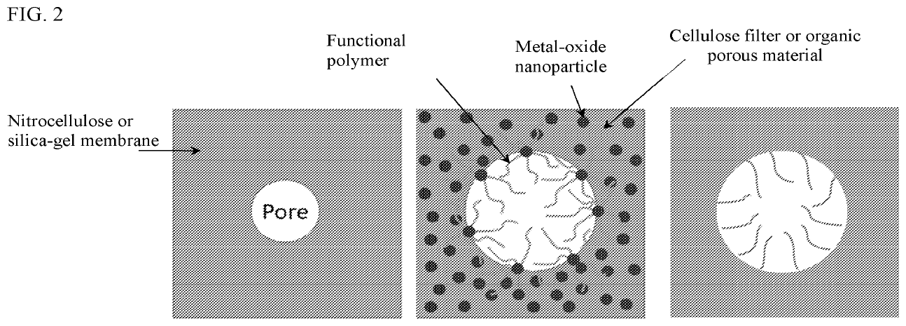

In some embodiments, disclosed methods include nanoparticle-mediated functionalization. This process includes functionalization of porous media or filters with metal oxide nanoparticles using, for example, a dip coating method, followed by immersion in an oxysilane precursor to form polysilane coating on the porous media (FIG. 1A). Such methods include embedding metal-oxide nanoparticles inside a cellulose membrane or filter paper, then using the nanoparticles as anchor points to covalently bind oxysilane polymers. FIG. 2 also depicts an illustration of the porous media that includes nanoparticles and organosilane compounds.

In some embodiments, disclosed methods include silanization of the filters directly using silicon-containing materials (organo-silicon compounds) described in FIG. 1B, without the use of nanoparticles. The terms “organosilicon” and “organo-silicon” are used interchangeably here to refer to any organic compound that contains silicon. This functionalization can be performed using a process referred to as “crossflow functionalization” to form covalent bonds between the oxysilane and the porous media. Possible polymer precursors can include, for example, silanes, alkoxysilane, siloxanes, oxysilanes, silsesquioxane in their different forms (monomers, polymers, dendrimers, hyperbranched and networks, physical and chemical gels), or any combination of form or material thereof. A single filter holder can contain filters functionalized with different silicon-based compounds.

The silanization of the filter material with silicon-based precursors can be done under any suitable conditions. Exemplary suitable conditions include, but are not limited to, functionalizing the filter material by exposing the filter to a precursor solution, referred to herein as a silanization solution. The silanization solution can contain a silane or oxysilane compound and a solvent (e.g., water and optionally an alcohol such as ethanol). The precursor concentration can be in the range 0.001 molar (M) to 5 M, in some embodiments from 0.01 M to 0.5 M. In some embodiments, the water/precursor molar ratio can be lower than 150, in some embodiments between 10 and 128, depending on the particular precursor, as well as other conditions. The ratio of solvent to precursor (e.g., water:precursor, [water+alcohol]:precursor, etc.) can be lower than 1000, in some embodiments in the range of 200-700. In other embodiments, however, the ratio of solvent to precursor can be in the range from 4 (approximately 20% precursor) to 99 (approximately 1% precursor) such as, for example, in the range of 5.67 (approximately 15% precursor) to 19 (approximately 5% precursor). The alcohol:precursor molar ratio should be in the range of 0 to 500, in some embodiments, around 200-500. In some embodiments, the water content can be in the range from 5%-15%. Silanization can result in a functional paper with high affinity to biological materials. Scanning electron microscopy images of the functionalized filter show homogenous coating of the filter with silanes (FIG. 3A before silanization and FIG. 3B shows after).

Porous media can also be functionalized with polymers such as amine-terminated polymers including, for example, polylysine or polyallylamine and other amine-containing monomers or their corresponding polymers such as ethyleneimine, oxazoline, and vinylpyridine, and other polymers containing amine groups in their main chains or sidechains.

The functionalization can be performed using crossflow functionalization to form electrostatic bonds between the functionalizing material and the porous media. For example, for the functionalization with polylysine, the polylysine concentration can be between 0.1 and 100 micromolar, and preferably between 1 and 10 micromolar.

Depending on the application, functionalization of porous media can also be performed with molecules or polymers that have other functional groups.

Disclosed herein are methods of forming porous media. One exemplary method is illustrated in FIG. 14. Some such embodiments are referred to herein as crossflow functionalization. Crossflow functionalization includes forced, and/or rapid passage or flow of the functionalization solution 12 across the porous media 14 using a pumping system (for example) in a way that allows rapid penetration of the solution throughout the pores of the media while avoiding precipitation. In some embodiments, where the porous media 14 is in the form of a sheet (e.g., some type of film, paper, or sheet type surface), this step can be described as forcing or drawing the functionalization solution across the sheet in the direction which 16 or in a direction parallel to the direction which the fluid sample is to be drawn through the porous sheet. For example, in the case of a porous cellulose filter paper, the functionalization solution may be drawn through the filter paper in the same or the opposite direction as the sample will flow through the paper.

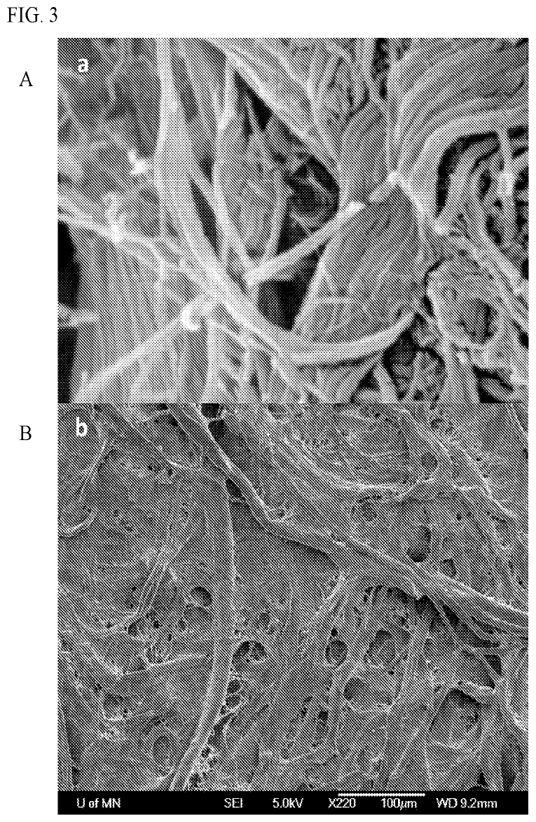

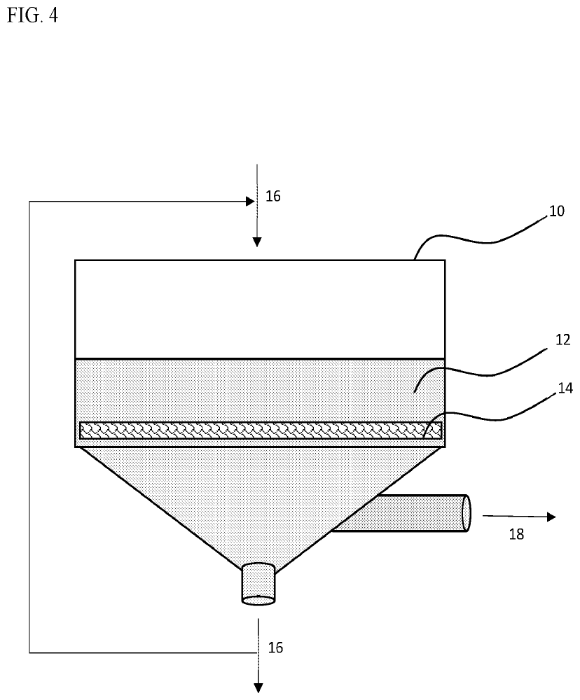

The functionalization can be conducted in a vessel, such as that depicted in FIG. 4. The vessel 10 can be made of glass, metal such as steel, or a polymer such as a plastic. By controlling the concentration of the functionalization solution 12, the flow rate, other factors, or combinations thereof, it is possible to control the thickness of the functionalized layer formed on the porous substrate. The functionalization can include the following steps: 1) fill the vessel with the functionalization solution; 2) force the solution, using a vacuum pump 18, for example, through/across the filter material allowing its efficient and rapid functionalization in 3-dimensional fashion; 3) recirculate the solution into the vessel to allow further functionalization with the same solution 16; 4) dry the functionalized porous media; 5) rinse the porous media; and 6) dry the porous media before use.

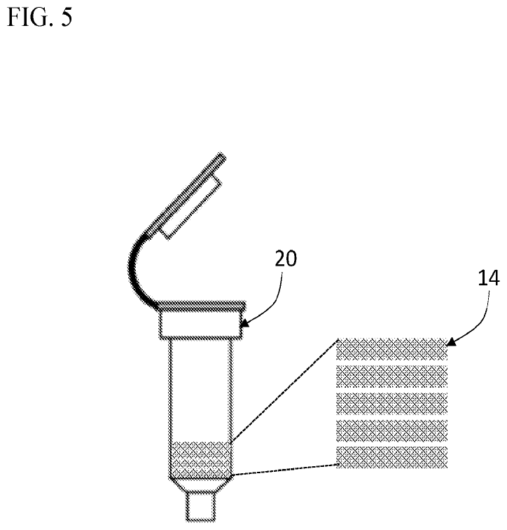

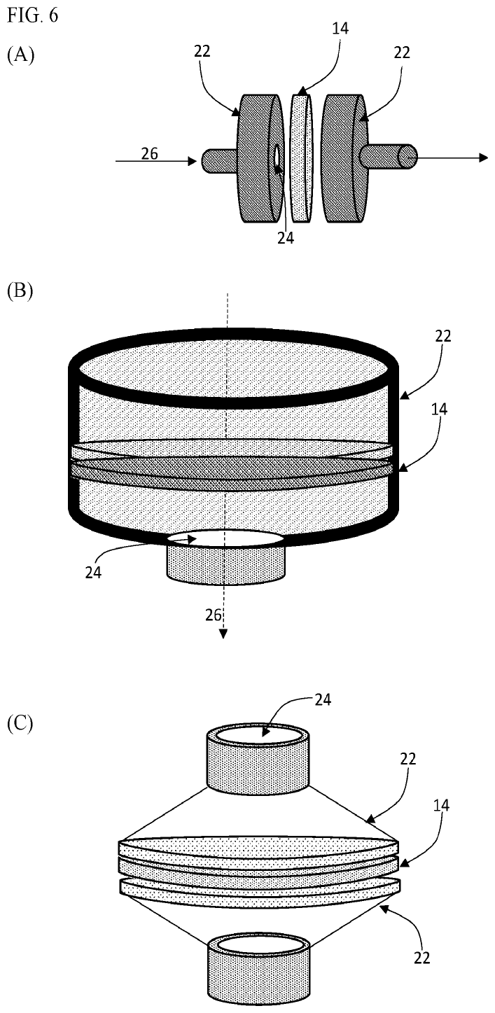

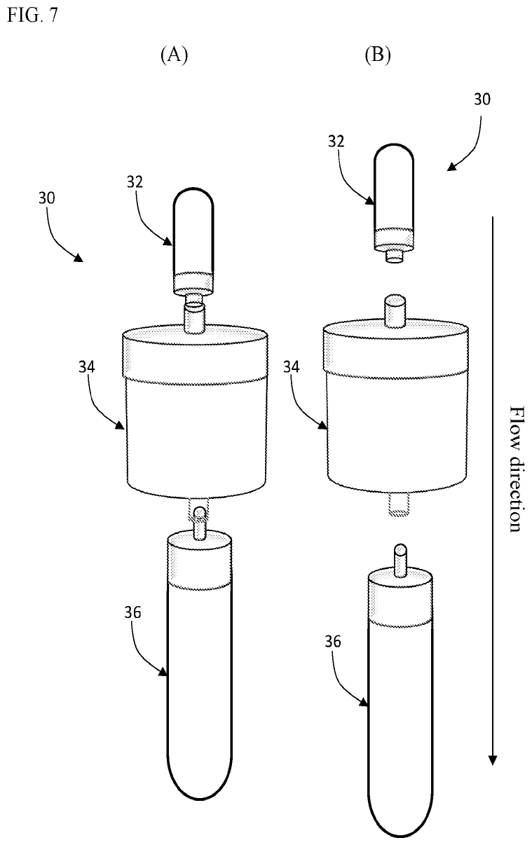

Disclosed filters can be utilized in various larger devices, including for example the illustrative different housing systems, filter holders, and components thereof, as shown in FIGS. 5, 6A-C, 7A, 7B, 8 and 9A-W. Disclosed illustrative devices can include devices such as spin columns. Spin columns can be used for filtration by centrifugation. The columns can be of different shapes or volumes (e.g., 5 uL to 100 mL), can have one or multiple functionalized filters with same or different material, with the same or different pore size and functionalizing agent. Disclosed components can include, for example filter holders that allow pumping fluids through the filters, or filter holders that allow sucking fluids through the filters by a vacuum pump or a water pump. Disclosed devices include devices such as that referred to herein as an eBiosampler (environmental sample Biosampler). The eBiosampler is a biocollection and bioextraction system used to process large volumes (FIGS. 7A, 7B and 8, for example). A sample workflow for a nucleic acid isolation using an eBiosampler is shown in FIG. 15.

In some embodiments, a biocollection and bioextraction system or “eBiosampler” device can be used. The illustrative eBiosampler depicted in FIGS. 7A,7B and 8 is a disclosed design that combines fluid collection of large volume samples, subsequent extraction, preservation, concentration, separation, or any combinations thereof of a target biological material.

Such systems can be used according to the following summarized steps. The fluid sampling process can include collecting a large volume of the sample (e.g., environmental water or circulating air), using a pump or syringe 62 to pass the total volume of the fluid sample through the eBiosampler 30, adding buffers, locking the filtration cartridge 34 with the sample inside for further use for the recovery and isolation of the eDNA. Recovery and isolation of eDNA may be accomplished in a lab, as opposed to the field, where the sample collection steps likely happen. The filtration cartridge 34 and the collection tube may be connected to perform multiple centrifugations with different buffers to enable one to isolate the target biological material. Isolated target biological material can be stored at −80° ° C. for further analysis with PCR (if the target biological material is a nucleic acid) or other techniques appropriate for analyzing the target biological material.

The systems should also be understood to be compatible with collecting biological material from gases such as air, or from fluid suspensions such as breath. Airborne DNA is an often overlooked source of information for a number of applications, including conservation, wildlife crime monitoring, and forensics.

The invention is defined in the claims. However, below there is provided a non-exhaustive listing of non-limiting exemplary aspects. Any one or more of the features of these aspects may be combined with any one or more features of another example, embodiment, or aspect described herein.

### EXEMPLARY EMBODIMENTS

Embodiment 1. A device for separating a biological entity from a fluid sample, the device comprising porous material comprising bound material, wherein the bound material functions to separate the biological entity from the fluid sample.
Embodiment 2. A device for separating a biological entity from a fluid sample, the device comprising porous material comprising bound nanoparticles bonded to a bound material, wherein the bound material functions to separate the biological entity from the fluid sample.
Embodiment 3. A method of forming filtration media, the method comprising:
providing a sheet of porous material having a matrix and pores within the matrix,
contacting the sheet of porous material with a functionalization solution comprising:
   an organic material, a biological entity, an inorganic material, or a combination thereof; and
   a solvent; and
forcing the functionalization solution through the pores of the porous material in a direction that is substantially parallel to the direction that a fluid sample is to be filtered using the filtration media.
   Embodiment 4. A device for separating a biological entity from a fluid sample, the device comprising:
at least one filter, the at least one filter comprising a sheet of porous material comprising bound material;
an inlet; and
an outlet,
wherein the fluid sample comprising the biological entity and a fluid matrix are introduced via the inlet of the filter, the fluid sample contacts the porous material of the filter wherein the bound material functions to separate the biological entity from the fluid matrix and the fluid matrix exits the device via the outlet.
   Embodiment 5. A method of separating a biological entity from a fluid sample, the method comprising:
contacting the fluid sample with a porous material, the porous material comprising a matrix having surface area and pores, the surface area of the porous material comprising bound material;
removing at least some of the fluid matrix from the porous material; and
retaining the biological entity within or in contact with the bound material.
   Embodiment 6. The device or method of any preceding Embodiment, wherein the biological entity comprises a biomolecule, a virus, a prion, a microorganism, a cell, a tissue, or a combination thereof.
   Embodiment 7. The device or method of any preceding Embodiment, wherein the biomolecule comprises DNA, RNA, a protein, a lipid, a saccharide, a biopolymer, or a combination thereof.
   Embodiment 8. The device or method of Embodiment 6, wherein the microorganism is a bacterium, a fungus, a mold, a virus, or a combination thereof.
   Embodiment 9. The device or method of any preceding Embodiment, wherein the biopolymer comprises a polypeptide, an oligopeptide, a polysaccharide, an oligosaccharide, or a combination thereof.
   Embodiment 10. The device or method of any preceding Embodiment, wherein the bound material comprises or is derived from Tetraethyl orthosilicate, Triethoxy(octyl)silane, Triethoxyoctylsilane, Dodecyltriethoxysilane, n-Octadecyltriethoxysilane, Hexadecyltrimethoxysilane, Triethoxy(ethyl)silane, Isobutyltriethoxysilane, Triethoxymethylsilane, Trimethoxy(octadecyl)silane, (3-Aminopropyl)triethoxysilane, (3-Aminopropyl)trimethoxysilane, (3-Glycidyloxypropyl) trimethoxysilane, Tetraethyl orthosilicate, 3-(Trimethoxysilyl)propyl methacrylate, (3-Aminopropyl)trimethoxysilane, (3-Mercaptopropyl)trimethoxysilane, (3-Glycidyloxypropyl)trimethoxysilane, Trimethoxy(propyl)silane, Trimethoxy(3,3,3-trifluoropropyl)silane, 3-[2-(2-Aminoethylamino)ethylamino]propyl-trimethoxysilane, [3-(2-Aminoethylamino)propyl]trimethoxysilane, Trichlorosilane, 1H,1H,2H,2H-Perfluorooctyltriethoxysilane, 1H,1H,2H,2H-Perfluorodecyltriethoxysilane, (3-Glycidyloxypropyl)trimethoxysilane, (3-Glycidyloxypropyl)triethoxysilane, or combinations thereof.
   Embodiment 11. The device or method of any preceding Embodiment, wherein the porous material comprises any material with an oxide containing surface.
   Embodiment 12. The device or method of any preceding Embodiment, wherein the porous material comprises cellulose, nitrocellulose, glass fiber, carbon, a metal oxide, or a combination thereof.
   Embodiment 13. The device or method of any preceding Embodiment, wherein the porous material has a porosity from 0.1 μm to 50 μm.
   Embodiment 14. The device or method of any one of Embodiments 1 to 12, wherein the porous material has a porosity from 0.5 μm to 15 μm.
   Embodiment 15. The device or method of any one of Embodiments 1 to 12, wherein the porous material has a porosity from 1 μm to 12 μm.
   Embodiment 16. The device or method of any one of Embodiments 4 to 15, wherein the device includes from 1 to 100 filters comprising porous material.
   Embodiment 17. The device or method of any one of Embodiments 4 to 15 wherein the device includes from 1 to 20 filters comprising porous material.
   Embodiment 18. The method of any one of Embodiments 3 or 6 to 17, wherein the functionalization solution comprises a solvent comprising water, an alcohol, or combinations thereof.
   Embodiment 19. The method of any one of Embodiments 3 or 6 to 18, wherein the functionalization solution has a concentration from 0.001 Molar to 5 Molar organo silane compound.
   Embodiment 20. The method of any one of Embodiments 3 or 6 to 19, wherein the functionalization solution has a concentration from 0.01 Molar to 0.5 Molar organo silane compound.
   Embodiment 21. The method of Embodiment 20, wherein the solvent comprises water and the molar ratio of water to organo silane material is less than 150.
   Embodiment 22. The method of Embodiment 20, wherein the solvent comprises water and the molar ratio of water to organo silane material is from 10 to 30.
   Embodiment 23. The method of Embodiment 20, wherein the solvent comprises water and at least one alcohol.
   Embodiment 24. The method of Embodiment 23, wherein the molar ratio of water plus alcohol to organo silane material is less than 1000.
   Embodiment 25. The method of Embodiment 23, wherein the molar ratio of water plus alcohol to organo silane material is from 200 to 700.
   Embodiment 26. The method of Embodiment 23, wherein the molar ratio of alcohol to organo silane material is from 0 to 500.
   Embodiment 27. The method of Embodiment 23, wherein the molar ratio of alcohol to organo silane material is from 200 to 500.
   Embodiment 28. The method of Embodiment 23, wherein the organo silane material to solvent ratio is from 1% to 20%.
   Embodiment 29. The method of Embodiment 23, wherein the organo silane material to solvent ratio is from 5% to 15%.
   Embodiment 30. The method of Embodiment 23, wherein the water content in the functionalization solution is from 5% to 15%.
   Embodiment 31. The method or device of any of Embodiments 1 to 30, wherein the porous material is functionalized with an amine terminated polymer.
   Embodiment 32. The method or device of Embodiment 31, wherein the amine terminated polymer is selected from polylysine or polyallylamine and other amine-containing monomers or their corresponding polymers such as ethyleneimine, oxazoline, and vinylpyridine, and other polymers containing amine groups in their main- or sidechains.
   Embodiment 33. The method or device of any one of Embodiments 1 to 32, wherein the amine terminated polymer has a concentration from 0.1 to 100 micromolar.
   Embodiment 34. The method or device of any one of Embodiments 1 to 33, wherein the amine terminated polymer has a concentration from 1 to 10 micromolar.
   Embodiment 35. The method or device of any of Embodiments 1 to 34, wherein the porous material or filters containing the porous material are housed within a columnar shaped device.
   Embodiment 36. The method or device of Embodiment 35, wherein the columnar shaped device is configured to accommodate liquid samples having volumes from 5 microliters to 1000 milliliters.
   Embodiment 37. The method or device of any of Embodiments 1 to 36, wherein the fluid sample is forced through the porous material using a vacuum pump.
   Embodiment 38. The method or device of any of Embodiments 1 to 37 further comprising a syringe, a collection tube, an air filtration unit, or a fluidic system connected to the inlet of the device.
   Embodiment 39. The method or device of any of Embodiments 1 to 38 further comprising a syringe, a collection tube, an air filtration unit, or a fluidic system connected to the outlet of the device.
   Embodiment 40. The method or device of any of Embodiments 1 to 39 further comprising at least one chamber disposed between the filter and the outlet.
   Embodiment 41. The method or device of Embodiment 40, wherein the at least one chamber contains a lysis solution, a bonding solution, a preservation solution, an extraction solution, a filtration solution, or any combination thereof.
   Embodiment 42. The method or device of Embodiment 40, comprising at least two chambers.
   Embodiment 43. The method or device of any one of Embodiments 1, 2, or 4 to 42, wherein the inlet is in fluid communication with a fluid injection system.
   Embodiment 44. The method or device of any one of Embodiments 1, 2, or 4 to 42, wherein the outlet is in fluid communication with a collection tube, centrifuge tube, a spin column, or any combination thereof.
   Embodiment 45. The method or device of any of Embodiments 1, 2 or 4 to 44, wherein the porous media is formed by passing a functionalization solution through a sheet of the porous media in a direction substantially parallel to the direction in which a fluid sample will be drawn through the sheet of the porous media.
   Embodiment 46. The method or device of Embodiment 45, wherein the functionalization solution is drawn through the sheet of porous media.
   Embodiment 47. The method or device of Embodiment 45, wherein the functionalization solution is forced through the sheet of porous media.
   Embodiment 48. The device of Embodiment 1, further comprising a collection chamber and spin column, wherein the collection chamber or the spin column or both comprise the porous material comprising bound material.
   Embodiment 49. The device of Embodiment 48, wherein the collection chamber comprises a sealable cap.
   Embodiment 50. The method or device of any one of Embodiments 1, 2, or 4-49, wherein the fluid sample comprises a liquid.
   Embodiment 51. The method or device of any one of Embodiments 1, 2, or 4-49, wherein the fluid sample comprises a gas.
   Embodiment 52. The device of any one of Embodiments 1, 2, 4, or 6 to 51, further comprising an external air filtration system couple to the device.

The present invention is illustrated by the following examples. It is to be understood that the particular examples, materials, amounts, and procedures are to be interpreted broadly in accordance with the scope and spirit of the invention as set forth herein.

### EXAMPLES

All reagents, starting materials, and solvents used in the following examples were purchased from commercial suppliers (such as Sigma Aldrich, St. Louis, MO) and were used without further isolation unless otherwise indicated.

### Example 1—Formation of Filter Media

Filter functionalization was performed by the crossflow functionalization process described herein. The choice of polymer depends on the targeted biomolecule and the buffers associated with its extraction and isolation. The filters used to recover free eDNA and those used to recover total eDNA (cells, tissues) and the filters stacked in spin columns to recover and purify nucleic acids from biological samples are all different.

To recover free eDNA, (3-Aminopropyl)triethoxysilane (APTES)/(AEEAS) was used (no need for lysis as the DNA is already free) and then isolation with spin columns.

To recover total eDNA, a strong lysis buffer such as guanidine thiocyanate (GTCT) was used.

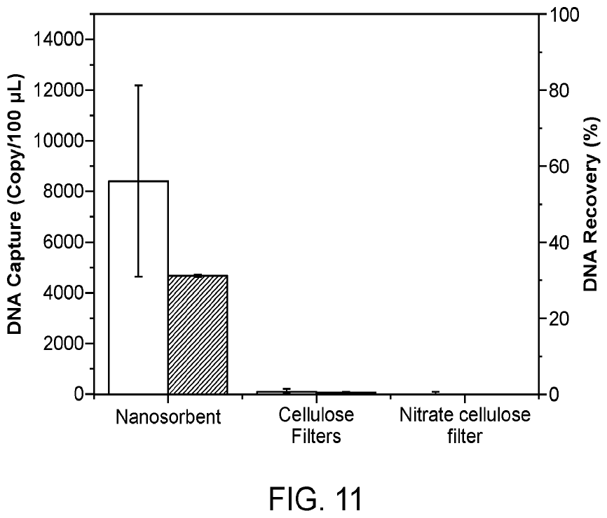

For free DNA, one or more filters were used to recover eDNA (see results in FIG. 10 and FIG. 11). The new eBiosampler allows stacking of multiple filters, thus allowing the efficiency to get closer to 100% capture, with an enhanced eDNA recovery). The stacked filters can have different pore sizes to improve the flow and the capture. This also will help limit clogging. After adding the adequate buffers, target recovery is performed in the reverse direction of the flow.

DNAeasy powersoil pro kit (the most used for eDNA), RNAeasy kit and Viral RNS kit was used for coronavirus detection from Qiagen.

### Example 2—Extraction of Environmental DNA (eDNA) from Environmental Samples

To evaluate the feasibility of the capture of viral eDNA, Koi Herpes virus was utilized as an analytical target in aquarium water in the presence of different fish species (FIG. 10 and FIG. 11). A filter prepared as in Example 1 was utilized to extract and detect nucleic acids (utilizing PCR amplification) from fishes and viruses (Koi herpes virus) (FIG. 10 and FIG. 11).

### Working Model (Strawberry DNA)

The DNA used in these experiments was extracted from strawberries. The DNA obtained was then diluted in deionized water to reach a concentration of 10 ng/ml. The DNA measurement was taken by Qubit 3. The DNA solution was filtered through one of three different filters: a filter prepared as described in Example 1, a normal cellulose filter, or a nitrocellulose filter.

The filters were inserted into a filter holder and connected to the fluidic system. The system consisted of a pump and a turbine that pumped water from the “DNA solution” and passed it through the filter. The objective was to measure the difference between the initial solution DNA concentration and the final concentration (filtrates).

The amount of DNA captured was calculated using the following equation:

$$DNA c \% = ( \frac{DNA i - DNA f}{DNA i} ) * 100$$

Where DNAc % is the percentage of the amount of [DNA] captured by the filter, [DNAi] is initial solution DNA concentrations, and finally [DNAf] is the final concentrations (filtrates).

The loading capacity of the captured DNA was calculated by accumulating [DNAc] during the filtration process, the measurements of [DNAc] were taken after each 50 millimeters of filtered solution.

The results are shown in FIG. 10.

### Working Model: KHV DNA

The DNA used in these experiments is extracted from Koi Herpesvirus KHV. The DNA obtained was then diluted in demineralized water (until the [DNA] to a concentration of 0.27 copies/μL. The [DNA] measurement is estimated by quantitative PCR (qPCR) in copy/μL.

The same volume (100 ml) of DNA solution ([DNA]=0.27 copies/μL) was passed through one of three different filters using a 50 ml syringe: a filter prepared as described in Example 1, a normal cellulose filter, or a nitrocellulose filter.

DNA was recovered from each filter using NaCl solution, then purified and eluted using the new spin columns disclosed here. The measurement of the quantity of DNA copy recovered/μL was conducted by qPCR.

The results depicted in FIG. 10 show that the filter of Example 1 reaches a loading capture of 100% of free viral DNA and recovers around 40%, while conventional cellulose and nitrocellulose filters showed insignificant capture and recovery (FIG. 11 and Table 1). The results presented in FIG. 11 show that the filter of Example 1 reaches a loading capacity for eDNA of 381 μg of eDNA per g of filter, which is over two orders of magnitude greater than conventional filters. This loading capacity means that a single filter of Example 1 can be used to filter up to 38 liters of water, depending on water quality and assuming an eDNA concentration in water of 10 ng/mL. Such performance could afford a capacity that was 300 times that of what was in the aquarium and is expected to significantly reduce the sampling effort and improve performance.

### Example 3—Extraction of SARS-COV-2 RNA from COVID-19 Patient Samples

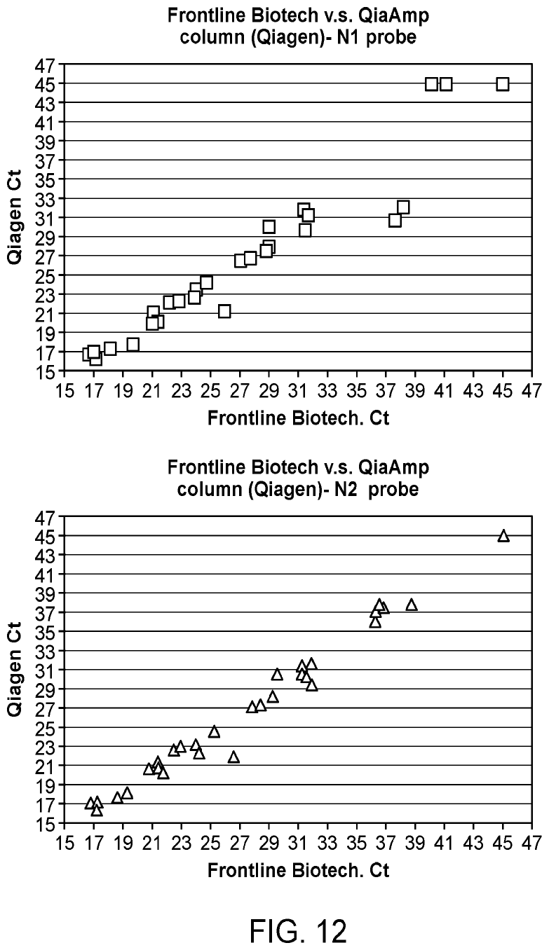

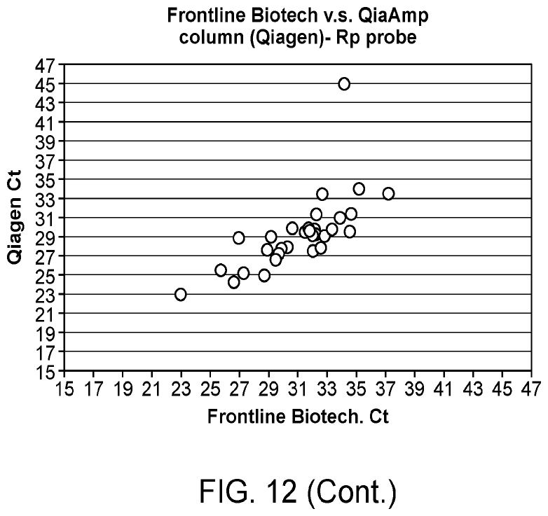

The filter of Example 1 was successfully applied to the extraction of SARS-COV-2 RNA from COVID-19 patients' samples (FIG. 12 and FIG. 13).

This test was performed by the COVID-19 Diagnostic Laboratory at the University of Minnesota.

As seen below, the disclosed filter performed better and was able to detect positive samples that were missed by commercially available products (QIAprep&amp Viral RNA UM Kit.—QIAGEN).

TABLE 1
Analytical performance of disclosed system vs. commercial kits

| | Example 1 | Conventional silica gel spin |
|---|---|---|
| Product | Filter | column (from Commercial) |
| Specificity | 100% | 100% |
| Sensitivity | >98 | >97% |
| Detection limit | <10 copies | 10-100 copies/mL |
| | per mL | |
| Sample volume | 5 μl-100 L | 5 μL-2 mL (spin column), |
| | | and 5 μL-50 μL (magnetic |
| | | beads) |
| Nucleic acid | >70% | 10-40% |
| recovery | | |

The results of the sensitivity and specificity studies are provided in Table 1 and FIG. 11. The results indicate a better sensitivity and specificity of the kits from the current invention as compared to commercial kits. In addition, FIG. 12 shows a linearity and agreement between the cycle threshold (Ct) values obtained with commercial kits and those obtained with disclosed methods and filters.

TABLE 2
Comparison of the clinical specificity
and sensitivity of the Example 1 filter

Combined results from a preliminary and
full-scale study (80 samples)

| | Commercial kits | Example 1 Filter |
|---|---|---|
| True Positives | 46 | 46 |
| False Positives | 5 | 0 |
| True negatives | 34 | 34 |
| False negatives | 6 | 3 |
| Sensitivity | 88.46 | 93.88 |
| Specificity | 87.18 | 100.00 |

Limit of Detection: To evaluate the limit of detection of the Filter of Example 1 and QIAamp Viral RNA UM Kit.—QIAGEN, different concentration of SARS-COV-2 RNA were prepared through a serial dilution, to obtain 90 copies/μL (Cp/μL), 45 Cp/μL, 15 Cp/μL, 5 Cp/μL, 1.67 Cp/μL, and 0.56 Cp/μL. A volume of 140 μL of the serially diluted solutions was prepared and was then passed through the commercial and Example 1 columns for RNA extraction, followed by reverse transcription PCR (RT-PCR)

The results are shown in FIG. 13 and Table 3 and show that the limit of detection (LOD) for both kits is around 1 Cp μL, within the 5% standard deviation or variation generally observed with different commercial kits.

TABLE 3
Limits of detection, reproducibility, and analytical sensitivity

| | Commercial kit | |
|---|---|---|
| | QIAamp Viral RNA | |
| | UM Kit. - QIAGEN | Example 1 |
| LOD (copies/μL) | 1 | 1 |
| Divergence factor | 1.63 | 0.74 |
| (lower values mean | | |
| higher reproducibility) | | |
| Analytical Sensitivity | 0.09 Ct value per 1 | 0.28 Ct value per 1 |
| | RNA copy/μL | RNA copy/μL |
| | OR | OR |
| | 11.1 RNA copies/μL | 3.5 RNA copies/μL |
| | per 1 Ct value | per 1 Ct value |

The term “comprises” and variations thereof do not have a limiting meaning where these terms appear in the description and claims.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “and/or” means one or all of the listed elements or a combination of any two or more of the listed elements; the terms “comprises,” “comprising,” and variations thereof are to be construed as open ended—i.e., additional elements or steps are optional and may or may not be present. Such terms will be understood to imply the inclusion of a stated step or element or group of steps or elements but not the exclusion of any other step or element or group of steps or elements. Unless otherwise specified, “a,” “an,” “the,” and “at least one” are used interchangeably and mean one or more than one.

As used herein, the term “or” is generally employed in its usual sense including “and/or” unless the content clearly dictates otherwise.

Any reference to standard methods (e.g., ASTM, TAPPI, AATCC, etc.) refer to the most recent available version of the method at the time of filing of this disclosure unless otherwise indicated.

Also herein, the recitations of numerical ranges by endpoints include all numbers subsumed within that range (e.g., 1 to 5 includes 1, 1.5, 2, 2.75, 3, 3.80, 4, 5, etc.).

Herein, “up to” a number (for example, up to 50) includes the number (for example, 50).

The term “in the range” or “within a range” (and similar statements) includes the endpoints of the stated range.

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

1. A device for separating a biological entity from a fluid sample, the device comprising:
porous material comprising bound material, wherein the bound material functions to separate the biological entity from the fluid sample.

2. The device of claim 1, wherein
the porous material comprises nanoparticles bound to the bound material, wherein the bound material functions to separate the biological entity from the fluid sample.

3. A method of forming filtration media, the method comprising:
providing a sheet of porous material having a matrix and pores within the matrix,
contacting the sheet of porous material with a functionalization solution comprising:
an organic material, a biological entity, an inorganic material, or a combination thereof; and
a solvent; and
forcing the functionalization solution through the pores of the porous material in a direction that is substantially parallel to the direction that a fluid sample is to be filtered using the filtration media.

4. (canceled)

5. A method of separating a biological entity from a fluid sample, the method comprising:
contacting the fluid sample with a porous material, the porous material comprising a matrix having surface area and pores, the surface area of the porous material comprising bound material;
removing at least some of the fluid matrix from the porous material; and
retaining the biological entity within or in contact with the bound material.

6. The device of claim 1, wherein the biological entity comprises a biomolecule, a virus, a prion, a microorganism, a cell, a tissue, or a combination thereof.

7. The device of claim 1, wherein the biomolecule comprises DNA, RNA, a protein, a lipid, a saccharide, a biopolymer, or a combination thereof.

8. (canceled)

9. The device of claim 1, wherein the biopolymer comprises a polypeptide, an oligopeptide, a polysaccharide, an oligosaccharide, or a combination thereof.

10. The device of claim 1, wherein the bound material comprises or is derived from Tetraethyl orthosilicate, Triethoxy(octyl)silane, Triethoxyoctylsilane, Dodecyltriethoxysilane, n-Octadecyltriethoxysilane, Hexadecyltrimethoxysilane, Triethoxy(ethyl)silane, Isobutyltriethoxysilane, Triethoxymethylsilane, Trimethoxy(octadecyl)silane, (3-Aminopropyl)triethoxysilane, (3-Aminopropyl)trimethoxysilane, (3-Glycidyloxypropyl) trimethoxysilane, Tetraethyl orthosilicate, 3-(Trimethoxysilyl)propyl methacrylate, (3-Aminopropyl)trimethoxysilane, (3-Mercaptopropyl)trimethoxysilane, (3-Glycidyloxypropyl)trimethoxysilane, Trimethoxy(propyl)silane, Trimethoxy(3,3,3-trifluoropropyl)silane, 3-[2-(2-Aminoethylamino)ethylamino]propyl-trimethoxysilane, [3-(2-Aminoethylamino)propyl]trimethoxysilane, Trichlorosilane, 1H,1H,2H,2H-Perfluorooctyltriethoxysilane, 1H,1H,2H,2H-Perfluorodecyltriethoxysilane, (3-Glycidyloxypropyl)trimethoxysilane, (3-Glycidyloxypropyl)triethoxysilane, or combinations thereof.

11. The device of claim 1, wherein the porous material comprises any material with an oxide containing surface.

12. The device of claim 1, wherein the porous material comprises cellulose, nitrocellulose, glass fiber, carbon, a metal oxide, or a combination thereof.

13-17. (canceled)

18. The method of claim 3, wherein the functionalization solution comprises a solvent comprising water, an alcohol, or combinations thereof.

19. The method of claim 3, wherein the functionalization solution has a concentration from 0.001 Molar to 5 Molar organo silane compound.

20-30. (canceled)

31. The device of claim 1, wherein the porous material is functionalized with an amine terminated polymer.

32-34. (canceled)

35. The device of claim 1, wherein the porous material or filters containing the porous material are housed within a columnar shaped device.

36. (canceled)

37. (canceled)

38. The device of claim 1, further comprising a syringe, a collection tube, an air filtration unit, or a fluidic system connected to the inlet of the device.

39. The device of claim 1, further comprising a syringe, a collection tube, an air filtration unit, or a fluidic system connected to the outlet of the device.

40. The device of claim 1, further comprising at least one chamber disposed between the filter and the outlet.

41. (canceled)

42. (canceled)

43. The device of claim 1, wherein the inlet is in fluid communication with a fluid injection system.

44. The device of claim 1, wherein the outlet is in fluid communication with a collection tube, centrifuge tube, a spin column, or any combination thereof.

45-49. (canceled)

50. The device of claim 1, wherein the fluid sample comprises a liquid.

51. The device of claim 1, wherein the fluid sample comprises a gas.

52. The device of claim 1, further comprising an external air filtration system couple to the device.
